(19) United States
(12) Patent Application Publication
(10) Pub. No.: US 2009/0191143 A1
(43) Pub. Date: Jul. 30, 2009

(54) HAIR SHAPING KIET AND PROCESS COMPRISING AT LEAST ONE NON-HYDROXIDE BASE

(75) Inventors: Gerard Malle, Villiers S/Morin (FR); Damien Lavergne, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/365,065

(22) Filed: Feb. 3, 2009

Related U.S. Application Data
(62) Division of application No. 10/989,087, filed on Nov. 16, 2004, now abandoned.
(60) Provisional application No. 60/562,217, filed on Apr. 15, 2004.

(30) Foreign Application Priority Data
Nov. 18, 2003 (FR) 0350852

(51) Int. Cl. A61K 8/64; A61Q 5/04

(52) U.S. Cl. 424/70.51

(57) ABSTRACT

The disclosure provides ready-to-use cosmetic compositions for permanently shaping keratin fibers comprising, as permanent hair-shaping active agent, a base not belonging to the hydroxide family. The disclosure also provides kits comprising compartments to be placed in contact to form the ready-to-use compositions, and processes using these compositions.

This application claims the benefit of U.S. Provisional Application No. 60/562,217, filed Apr. 15, 2004, which is hereby incorporated by reference.

Disclosed herein are ready-to-use cosmetic compositions for permanently shaping keratin fibers, comprising, as a permanent hair-shaping active agent, at least one base not belonging to the hydroxide family. The disclosure also provides kits comprising compartments to be placed in contact to form the ready-to-use compositions, and processes using these compositions.

The term “keratin fibers,” as used herein, means fibers of human or animal origin such as head hair, other body hairs, the eyelashes, wool, angora, cashmere, fur, and the like. Throughout the disclosure, reference is made to head hair, although the presently claimed invention is not limited to keratin fibers or head hair.

The term “permanent shaping,” as used herein, includes the curling, permanent shaping or setting of Caucasian, Asiatic, and North African hair.

The term “bases not belonging to the hydroxide family,” as used herein, means organic or inorganic bases containing no hydroxide ions in their chemical formula and capable of accepting a proton. These bases not belonging to the hydroxide family may be used as beta-elimination active agents resulting in lanthionization, as described further below.

The term “base,” as used herein, means a compound capable of accepting a proton.

The expression “between A and B,” as used herein, means ranging from A to B, the limits A and B being included.

Two techniques are used to permanently reshape the hair. They are based on breaking the cystine disulfide bonds present in keratin.

The first technique comprises opening the disulfide bonds in a first step by means of a composition containing a reducing agent, and then, after having optionally rinsed the hair, in reconstituting, in a second step, the disulfide bonds by applying to the hair, which has been placed under tension beforehand with curlers or the like, or shaped or smoothed out by other means, an oxidizing composition also known as a fixing composition, so as to give the head of hair the desired shape. This technique makes it possible either to permanently shape the hair or to relax it, decurl it, or smooth it out.

The second technique comprises performing a lanthionization operation, using a composition containing a base belonging to the hydroxide family. This leads to replacement of the disulfide bonds (—CH₂—S—S—CH₂—) with lanthionine bonds (—CH₂—S—CH₂—). This lanthionization operation involves two consecutive chemical reactions:
The first reaction comprises a beta-elimination on the cystine brought about by a hydroxide ion, leading to the breaking of the disulfide bond and the formation of dehydroalanine.

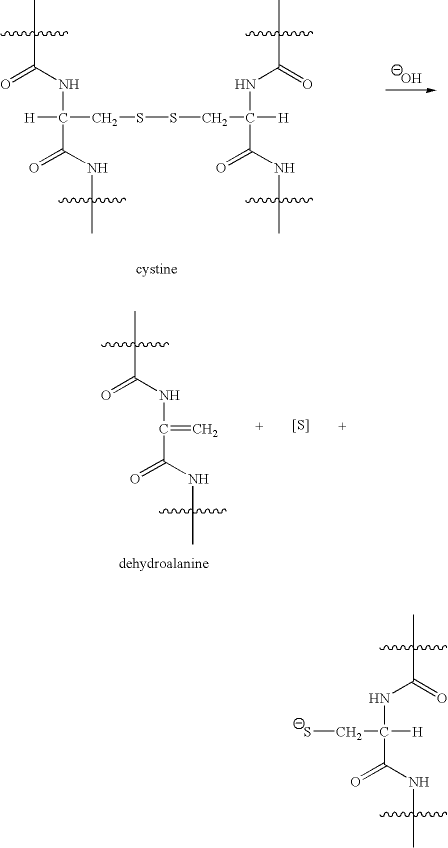

The second reaction is a reaction of the dehydroalanine with a thiol group. Specifically, the double bond of the dehydroalanine formed is a reactive double bond. It can react with the thiol group of the cysteine residue that has been released to form a new bond, referred to as a lanthionine bridge or bond or residue.

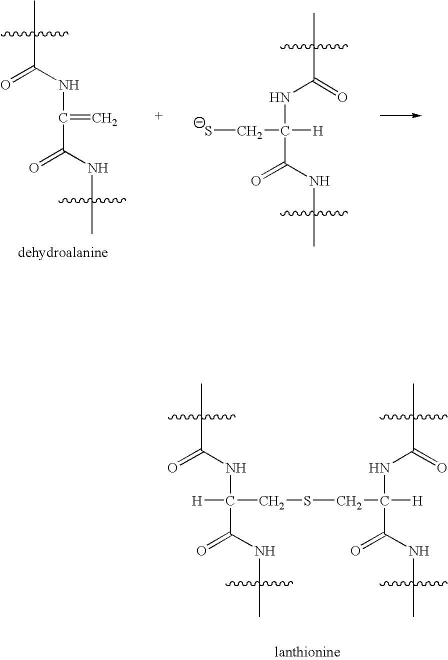

Relative to the first technique using a reducing agent, the lanthionization technique does not require a fixing step, since the formation of the lanthionine bridges is irreversible. It thus takes place in a single step and makes it possible either to make the hair wavy or to relax it, decurl it or smooth it out. However, it is mainly used to relax naturally curly hair.

For the first technique, the reducing compositions generally used for the first step of a permanent-waving or relaxing operation contain thiols, sulfites or bisulfites as reducing agents. These agents are generally used in essentially aqueous medium at concentrations of between 0.5 and 1M to obtain good opening of the disulfide bonds. Commonly used thiols include thioglycolic acid, cysteamine, glyceryl monothioglycolate, thiolactic acid, and cysteine. Thioglycolic acid is efficient at reducing the disulfide bonds of keratin at alkaline pH, for example, in the form of ammonium thioglycolate, and is the product most commonly used in permanent-waving (hair waving). However, it has been found that thioglycolic acid must be used in a sufficiently basic medium (in practice at a pH ranging from 8.5 to 9.5) if it is desired to obtain curling of sufficient intensity. In addition to the drawback of releasing an unpleasant odor requiring the use of fragrances to mask the odors, the use of a thiol at alkaline pH also leads to degradation of the fibers and to impairment of the artificial colorations.

Sulfites or bisulfites are also used for relaxing. They have drawbacks similar to thiols, with lower efficacy.

Thiols and sulfites (or bisulfites) also have the drawback of having poor stability in aqueous solution.

In general, the durability of the reshaping effects obtained with thiols and sulfites by reduction of disulfides followed by fixing is considered inferior to that which may be obtained via the lanthionization technique.

For the second technique, the compositions generally used to perform the lanthionization contain as base a hydroxide such as sodium hydroxide, guanidinium hydroxide and lithium hydroxide. These lanthionization active agents, which make it possible to open the disulfide bonds via a beta-elimination mechanism, are generally used in water-in-oil emulsions at concentrations ranging from 0.4M to 0.6M. They generally act within 10 to 15 minutes at room temperature. Sodium hydroxide is the agent most commonly used. Guanidinium hydroxide has recently been more commonly used in many such compositions. These two hydroxides, sodium hydroxide and guanidinium hydroxide, are the two main agents used for relaxing or decurling naturally curly hair. They have several advantages over ammonium thioglycolate and sulfites, for example, the absence of unpleasant odors, a shorter treatment time because only one implementation step is required, and greater durability and efficacy of the hair reshaping.

However, a major drawback of these hydroxides is their causticity. This causticity affects the scalp by causing irritation that may be severe. This may be partially overcome by the prior application to the scalp of a greasy protective cream often referred to as a “base” or “base cream,” the word “base” used here not having the meaning of a basic agent in the chemical sense. When the protective cream is combined with the hydroxide in a single composition, it is generally referred to as a “no-base” composition. The “no-base” technology is more convenient.

The causticity of the hydroxides also affects the state of the hair by giving it a coarse feel and making it much more fragile. The fragility may make the hair fray, break, or even dissolve if the treatment is prolonged. In some cases, hydroxides also cause decoloration of the natural color of the hair.

Formulations containing sodium hydroxide are generally referred to as “lye relaxers” and those not containing sodium hydroxide are generally referred to as “no-lye relaxers.”

The “no-lye” relaxing formulations often use guanidinium hydroxide. Since guanidinium hydroxide can be unstable, it is typically generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate makes the final rinsing of the hair much more difficult and leaves on the hair and scalp mineral particles that give it a coarse feel and an unaesthetic appearance resembling dandruff. The recent success of guanidinium hydroxide (“no-lye”) over sodium hydroxide (“lye”) appears to arise from better relaxing efficacy and better skin tolerance. However, these technologies using bases of the hydroxide family remain very aggressive for the hair and the scalp and require very strict control of the application time to avoid excessive irritation and impairment of the hair that may go as far as breaking. This aggressiveness arising from the causticity of hydroxides is just reason for these compositions for the lanthionization of the hair not to be used for permanent-waving (hair waving), but more often reserved for relaxing (hair straightening or hair relaxing).

Furthermore, hydroxides are known to be good agents for hydrolyzing amide functions (see, for example, March's Advanced Organic Chemistry, 5th Edition, Wiley Interscience, New York, “Hydrolysis of Amides” pages 474 et seq.), which thus lead to the breaking of peptide bonds by direct nucleophilic attack. The impairments observed in the case of the hair and keratin materials in the broad sense may be largely due to partial hydrolysis of the amide bonds of keratin.

In light of the above problems, there remains a real need for permanent hair shaping compositions that are less aggressive to the hair and the skin, including the scalp.

Various studies have been performed to attempt to simultaneously overcome the drawbacks of reducing agents (used in the first technique) and/or of hydroxides (used in the second technique).

Many reducing agents have been proposed to replace thioglycolic acid, but thioglycolic acid in its ammonium thioglycolate form remains both the reference compound and the compound most widely used in cosmetic formulations for permanently shaping the hair.

However, the use of reducing agents results in inadequate durability for permanent shaping, meaning that treatment must be repeated frequently and that there are inevitably instances of damage as treatment is repeated. In addition, the causticity of hydroxides limits their use strictly to the field of hair relaxing. No study has proposed a base as a permanent hair-shaping active agent that is generally both effective and less aggressive than soda lye or hydroxides.

After extensive studies, it has now been discovered, entirely surprisingly and unexpectedly, that the first step of the lanthionization process can be performed with bases not belonging to the hydroxide family. Excellent results in terms of intensity of permanent shaping, cosmetic and mechanical qualities of the hair, are thus obtained, with a durability greater than is obtained with ammonium thioglycolate, the reference product for permanent shaping.

Thus, in one aspect, the present disclosure provides cosmetic compositions comprising, in a cosmetically acceptable medium, at least one base not belonging to the hydroxide family, the cosmetically acceptable medium and the base being chosen such that the base not belonging to the hydroxide family is capable of reacting with the cystines of the keratin fibers, via a beta-elimination reaction, to produce dehydroalanine and lead to the formation of lanthionine, to produce curls on the keratin fibers ranging from 0.2 to 3 cm in diameter in less than 60 minutes.

The permanent shaping time may be less than 40 minutes, such as less than 30 minutes.

An exemplary beta-elimination active agent resulting in lanthionization is quinuclidine (1-azabicyclo[2.2.2]octane, Registry Number [100-76-5]).

The at least one base not belonging to the hydroxide family may be present in the cosmetic compositions in a molar concentration ranging from 0.01M to 4M, corresponding to concentrations ranging from 0.1% to 80% by weight relative to the total weight of the composition. For example, the at least one base may be present in concentrations ranging from 0.05M to 4M, corresponding to concentrations ranging from 0.5% to 80% by weight relative to the total weight of the composition.

The pH of the compositions may range from 9.6 to 14, for example, from 11 to 13.

In some embodiments, the at least one base not belonging to the hydroxide family is the only permanent hair-shaping active agent in the cosmetic composition, i.e., there is 0% of a base belonging to the hydroxide family.

In some embodiments, the compositions according to the present disclosure further comprise known reducing agents, for example, thioglycolic acid and thiolactic acid, and ester and amide derivatives thereof, including glyceryl monothioglycolate, cysteamine and its C₁-C₄ acyl derivatives such as N-acetylcysteamine or N-propionylcysteamine, cysteine, N-acetylcysteine, thiomalic acid, pantethine, 2,3-dimercaptosuccinic acid, sulfites or bisulfites of an alkali metal or alkaline-earth metal, the N-(mercaptoalkyl)-ω-hydroxyalkylamides described in Patent Application EP-A-354,835, the N-mono- or N,N-dialkylmercapto-4-butyramides described in Patent Application EP-A-368,763, the aminomercaptoalkylamides described in Patent Application EP-A-432,000, the N-(mercaptoalkyl)succinamic acid and N-(mercaptoalkyl)succinimide derivatives described in Patent Application EP-A-465,342, the alkylamino mercaptoalkylamides described in Patent Application EP-A-514,282, the azeotropic mixture of 2-hydroxypropyl thioglycolate and of (2-hydroxy-1-methyl)ethyl thioglycolate described in Patent Application FR-A-2,679,448, the mercaptoalkylaminoamides described in Patent Application FR-A-2,692,481, the N-mercaptoalkylalkanediamides described in Patent Application EP-A-653,202 and the formamidinesulfinic acid derivatives described in PCT Publication No. WO 02/39965, filed by Applicants.

When the compositions comprise at least one reducing agent, the agent may be present in a maximum concentration of up to 20% by weight, for example, in a concentration ranging from 0.1% to 10% by weight relative to the total weight of the composition.

The compositions may also comprise known hydroxides including alkali metal or alkaline-earth metal or transition metal or organic hydroxides such as sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide, and quaternary ammonium hydroxides.

When the compositions comprise at least one hydroxide, the hydroxide may be present in a concentration ranging from 0.01% to 3.5% by weight, for example, in a concentration ranging from 0.05% to 1.5% by weight relative to the total weight of the composition.

In some embodiments, the compositions contain 0% of base belonging to the hydroxide family, for example, alkali metals, alkaline-earth metals, transition metals, and organic hydroxides.

In certain embodiments, the compositions may also comprise at least one surfactant chosen from nonionic, anionic, cationic, and amphoteric surfactants, for example, alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and other nonionic hydroxypropyl ether surfactants.

When the cosmetic compositions contain at least one surfactant, the surfactant may be present in a maximum concentration of 30% by weight, for example ranging from 0.5% to 10% by weight relative to the total weight of the composition

The basic compositions may also comprise at least one treating agent chosen from cationic, anionic, nonionic and amphoteric treating agents to improve the cosmetic properties of the hair or to attenuate or avoid its degradation.

Treating agents that may be used include those described in French Patent Nos. 2,598,613 and 2,470,596. It is also possible to use as treating agents volatile or non-volatile, linear or cyclic silicones and mixtures thereof, polydimethylsiloxanes, quaternized polyorganosiloxanes such as those described in French Patent Application No. 2,535,730, polyorganosiloxanes comprising aminoalkyl groups modified with alkoxycarbonylalkyl groups, such as those described in U.S. Pat. No. 4,749,732, polyorganosiloxanes such as a Dimethicone Copolyol polyoxyalkyl polydimethylsiloxane copolymer, a polydimethylsiloxane containing stearoxy end groups (stearoxy dimethicone), a dialkylammonium acetate polydimethylsiloxane or a polydimethylsiloxane polyalkylbetaine copolymer described in British Patent No. 2,197,352, polysiloxanes organomodified with mercapto or mercaptoalkyl groups, such as those described in French Patent No. 1,530,369 and in European Patent Application No. 295,780, and also silanes such as stearoxytrimethylsilane.

The basic compositions may also contain other treating ingredients such as cationic polymers, for example those used in the compositions of French Patent Nos. 79/32,078 (2,472,382) and 80/26,421 (2,495,931); ionene cationic polymers, such as those used in the compositions disclosed in Luxembourg Patent No. 83,703; basic amino acids, such as lysine and arginine; acidic amino acids, such as glutamic acid and aspartic acid; peptides and peptide derivatives; protein hydrolysates; waxes; swelling agents; penetrating agents; agents for reinforcing the efficacy of the reducing agent, such as a SiO₂/PDMS (polydimethylsiloxane) mixture; dimethylisosorbitol; urea and urea derivatives; pyrrolidone; N-alkylpyrrolidones; thiamorpholinone; alkylene glycol and dialkylene glycol alkyl ethers, for example, propylene glycol monomethyl ether; dipropylene glycol monomethyl ether; ethylene glycol monoethyl ether and diethylene glycol monoethyl ether; C₃-C₆ alkanediols, for example propane-1,2-diol, propane-1,3-diol, and butane-1,2-diol; 2-imidazolidinone; other compounds such as fatty alcohols and lanolin derivatives; active ingredients such as pantothenic acid; agents for preventing hair loss; antidandruff agents; thickeners; suspending agents; sequestering agents; complexing agents; opacifiers; sunscreens, fragrances; and preserving agents.

In some embodiments of the compositions disclosed herein, the at least one base not belonging to the hydroxide family is capable of curling keratin fibers without being placed in contact beforehand with an organic solvent.

In some embodiments, the compositions are in aqueous form, for example, in the form of a thickened or unthickened lotion, a cream, or a gel.

In certain embodiments, the compositions may further comprise solvents such as ethanol, propanol, isopropanol, butanol, and glycerol. The maximum concentration of these solvents is 20% relative to the total weight of the composition. The cosmetically acceptable medium of the presently disclosed compositions may be water or an aqueous-alcohol solution of a C₂-C₈ alcohol, such as ethanol and isopropanol.

The compositions disclosed herein can also be in the form of thickened creams so as to hold the hair in the final desired shape during the leave-in time. These creams are made in the form of “heavy” emulsions, for example, emulsions based on glyceryl stearate, glycol stearate, self-emulsifying waxes, and/or fatty alcohols.

Liquids and gels comprising thickeners, such as carboxyvinyl polymers or copolymers that “stick” the hairs together and hold them in a desired shape during the leave-in time, may also be used.

The compositions also further comprise at least one adjuvant chosen from silicones in soluble, dispersed or microdispersed form; nonionic, anionic, cationic and amphoteric surfactants; ceramides, glycoceramides and pseudoceramides; vitamins and provitamins including panthenol; plant, animal, mineral, and synthetic oils; waxes other than ceramides, glycoceramides and pseudoceramides; water-soluble and liposoluble, silicone-based or non-silicone-based sunscreens; nacreous agents and opacifiers; sequestering agents; plasticizers; solubilizers; acidifying agents; mineral and organic thickeners; antioxidants; hydroxy acids; penetrating agents; fragrances; and preserving agents.

The present disclosure also provides kits comprising at least two compartments, wherein one of the compartments (i) comprises, in a cosmetically acceptable medium, at least one base not belonging to the hydroxide family, which is capable of reacting with the cystines of keratin fibers, via a beta-elimination reaction to produce dehydroalanine and lead to the formation of lanthionine, to produce with keratin fibers curls ranging from 0.2 to 3 cm in diameter in less than 60 minutes.

In certain embodiments, the kits further comprise an additional composition (ii) for caring for, conditioning, making up, removing makeup from, protecting, or cleansing (washing) keratin fibers.

The compositions of the kits may be packaged in separate compartments, containers or devices, optionally accompanied by suitable application means, which may be identical or different, such as fine brushes, coarse brushes, and sponges.

The present disclosure also provides processes for permanently shaping keratin materials using a cosmetic composition comprising, in a cosmetically acceptable medium, at least one base not belonging to the hydroxide family, the cosmetically acceptable medium and the base being chosen such that the base not belonging to the hydroxide family reacts with the cystines of the keratin fibers, via a beta-elimination reaction to produce dehydroalanine and lead to the formation of lanthionine, to produce with the keratin fibers curls ranging from 0.2 to 3 cm in diameter in less than 60 minutes.

In some embodiments, the permanent shaping time is less than 40 minutes, for example, less than 30 minutes.

In the permanent shaping processes, the compositions are applied to hair which is dry or has been wet beforehand, wound beforehand onto rollers (curlers), having a diameter ranging from 0.2 to 3.0 cm, it being possible to apply the composition as the hair is wound up; the composition is then left to act for a period ranging from 5 to 60 minutes, for example, 5 to 40 minutes. After the rollers have been removed, the hair is rinsed abundantly.

After applying the composition to the head of hair in the permanent shaping processes, the keratin fibers may be subjected to a heat treatment by heating to a temperature ranging from 30 to 60° C. In practice, the heat treatment may be performed using a hairstyling hood, a hairdryer, an infrared ray dispenser, and other standard heating devices.

It is also possible to use, as a means of both heating and shaping the hair, a hot iron at a temperature ranging from 60 to 220° C., for example, from 120 to 20° C.

The present disclosure also provides active agents which are bases not belonging to the hydroxide family for permanently shaping keratin fibers and uses thereof.

In some embodiments, these active agents for permanently shaping keratin fibers, act by means of a beta-elimination reaction producing dehydroalanine and leading to the formation of lanthionine.

The invention is illustrated in greater detail by the non-limiting examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

A simplified permanent-waving composition containing quinuclidine at a concentration of 1M in water, as permanent-waving active agent was prepared. The pH of the composition was 12.5. This composition was applied to Caucasian hair, wound beforehand onto curlers, and left on the hair for 30 minutes at a temperature of 50° C. The hair was rinsed and dried. The hair was beautifully curly and felt soft.

### EXAMPLE 2

A simplified permanent-waving composition containing quinuclidine at a concentration of 0.1M in water, as permanent-waving active agent was prepared. The pH of the composition was 12.1. This composition was applied to Caucasian hair, wound beforehand onto curlers, and left on the hair for 40 minutes at a temperature of 50° C. The hair was rinsed and dried. The hair was beautifully curly and felt soft.

1-10. (canceled)

11. A kit for producing keratin fiber curls ranging from 0.2 to 3 cm in diameter in less than 60 minutes comprising at least two compartments, wherein the at least one first compartment comprises,
a cosmetically acceptable medium,
at least one base not belonging to the hydroxide family, being chosen such that it is reactive with the cystines of the keratin fibers, via a beta-elimination reaction to produce dehydroalanine and lead to the formation of lanthionine, and it is the sole active Permanent shaping agent, and
at least one reducing agent present in the composition in an amount ranging from 0.1% to 10% by weigh relative to the total weight of the composition; and
further wherein the contents of the kit, when applied, produce keratin fiber curls ranging from 0.2 to 3 cm in diameter in less than 60 minutes.

12. The kit according to claim 11, wherein the at least one second compartment comprises at least one composition chosen from compositions for caring for, conditioning, making up, removing makeup from, protecting, cleansing and washing keratin fibers.

13. A process for permanently shaping keratin materials comprising
applying a cosmetic composition to the keratin fibers comprising,
a cosmetically acceptable medium,
at least one base not belonging to the hydroxide family being chosen such that it reacts with the cystines of the keratin fibers via a beta-elimination reaction, producing dehydroalanine and leading to the formation of lanthionine, and it is the sole active permanent shaping agent; and
at least one reducing agent present in an amount ranging from 0.1% to 10% by weigh relative to the total weight of the composition;
winding the keratin fibers around rollers ranging from 0.2 to 3 cm in diameter, either before or after applying the composition;
leaving the composition on the keratin fibers for a Permanent shaping time of less than 60 minutes; and
rinsing the keratin fibers.

14. The process for permanently shaping keratin materials according to claim 13, further comprising subjecting the keratin fibers to a heat treatment by heating to a temperature ranging from 30 to 60° C.

15. The process for permanently shaping keratin materials according to claim 13, wherein the heat treatment comprises heating with a hot iron at a temperature ranging from 60 to 220° C.

16. The process for permanently shaping keratin materials according to claim 15, wherein the hot iron is at a temperature ranging from 120 to 200° C.

17. The process for permanently shaping keratin materials according to claim 13, wherein the permanent shaping time is less than 40 minutes.

18. The process for permanently shaping keratin materials according to claim 17, wherein the permanent shaping time is less than 30 minutes.

19. An active agent for permanently shaping keratin fibers, wherein said active agent comprises,
at least one base not belonging to the hydroxide family being chosen such that it is reactive with the cystines of the keratin fibers, via a beta-elimination reaction to produce dehydroalanine and lead to a formation of lanthionine to permanently shape the keratin fibers, and it is the sole active Permanent shaping agent.

20. The process for permanently shaping keratin materials according to claim 13, wherein the at least one base not belonging to the hydroxide family is present in a concentration ranging from 0.01 M to 4M.

21. The process for permanently shaping keratin materials according to claim 20, wherein the at least one base not belonging to the hydroxide family is present in a concentration ranging from 0.05 M to 4M.

22. The process for permanently shaping keratin materials according to claim 13, wherein the pH of the cosmetic composition ranges from 9.6 to 14.

23. The process for permanently shaping keratin materials according to claim 22, wherein the pH of the cosmetic composition ranges from 11 to 13.

24. The process for permanently shaping keratin materials according to claim 13, wherein the cosmetic composition contains 0% of base belonging to the hydroxide family.

25. The process for permanently shaping keratin materials according to claim 13, wherein the cosmetic composition further comprises at least one adjuvant chosen from silicones in soluble, dispersed or microdispersed form; nonionic, anionic, cationic and amphoteric surfactants; ceramides, glycoceramides and pseudoceramides; vitamins and provitamins; plant, animal, mineral and synthetic oils; waxes other than ceramides, glycoceramides and pseudoceramides; water-soluble and liposoluble, silicone-based and non-silicone-based sunscreens; nacreous agents and opacifiers; sequestering agents; plasticizers; solubilizers; acidifying agents; mineral and organic thickeners; antioxidants; hydroxy acids; penetrating agents; fragrances; and preserving agents.

26. The process for permanently shaping keratin materials according to claim 13, wherein the at least one base not belonging to the hydroxide family is quinuclidine.
